(19) United States
(12) Patent Application Publication
(10) Pub. No.: US 2014/0275951 A1
(43) Pub. Date: Sep. 18, 2014

(54) GUIDEWIRE CONCIERGE

(71) Applicant: VOLCANO CORPORATION, San Diego, CA (US)

(72) Inventor: Bret Millett, Folsom, CA (US)

(73) Assignee: VOLCANO CORPORATION, San Diego, CA (US)

(21) Appl. No.: 14/208,431

(22) Filed: Mar. 13, 2014

Related U.S. Application Data
(60) Provisional application No. 61/778,732, filed on Mar. 13, 2013.

(51) Int. Cl. A61B 17/34; A61B 5/06; A61M 25/09

(52) U.S. Cl. CPC A61B 17/3415; A61B 17/3423; A61M 25/09041; A61B 5/061
USPC 600/407; 606/108

(57) ABSTRACT

The invention provides a method of intravascular intervention that includes inserting a catheter comprising an extended body into a lumen within tissue of a patient, advancing the catheter to a treatment site, vibrating a proximal end of the catheter with a mechanical vibrator, and treating the treatment site while a distal end of the catheter is vibrating.

This application claims the benefit of, and priority to, U.S. Provisional Patent Application No. 61/778,732, filed Mar. 13, 2013, which is incorporated by reference.

### FIELD OF THE INVENTION

The invention generally relates to systems and methods for intravascular intervention.

### BACKGROUND

When people suffer from plaque buildup in the arteries (“hardened arteries”), they are increased risk of deadly heart attacks and strokes. Heart attacks can be caused by the slow buildup of atherosclerotic plaque inside the blood vessels. The buildup of plaque occludes the flow of blood, and thus nutrients and oxygen, to a person's tissue and brain. Sometimes chunks of the atherosclerotic plaque break away and flow through the person's blood vessels. This can lead to serious and deadly strokes and heart attacks.

Some approaches to diagnosing or treating plaque buildup involve intravascular catheterization procedures. In these procedures, a guidewire is inserted into a patient's blood vessels and guided to the affected site. The guidewire is then used to guide imaging or treatment instruments. Unfortunately, once a guidewire has been inserted into the body, withdrawal of the guidewire exposes the tip to debris from the outside environment (e.g., blood and other contaminants). This debris can interfere with imaging when the guidewire is connected to another device. Debris from the outside environment also raises risks of infection and complication.

### SUMMARY

The invention relates to a device that receives a guidewire and keeps a tip of the guidewire with in the device at all times. This “concierge” device also allows a catheter to be slipped over the guidewire, allows the guidewire to be removed from the catheter, and allows a cable of a interventional device to be inserted into the catheter all within the device. In this manner, the guidewire tip and other parts are never exposed to the outside environment once an intravascular catheterization procedure is underway. The guidewire, once inside the concierge device, can then be connected to an interface cable or sheathed inside a catheter, all inside the concierge. Additionally, the concierge device can serve as a housing for connections between the guidewire and the intravascular imaging or treatment system. The device provides a dock in which to load and unload catheters. Intravascular imaging instruments and their associated components (e.g., a fiber-optic rotary joint) can be housed or connected to via the device. Thus the concierge device provides an apparatus for protecting a guidewire tip from contamination/debris when connecting the guidewire to other devices. The concierge device works by providing an environment in which the guidewire tip, once inserted into the apparatus, is never again exposed to an outside environment, even when connecting the guidewire to an interface cable.

Schematically, the concierge device can be thought of as a housing in which the housing lumen splits into various branches. The guidewire is inserted through one branch, the interface cable in another, and the catheter in yet another, with a common point of intersection for the branches. This allows for all connections to be made inside the concierge without having to expose the tip of the guidewire to the outside environment.

The concierge device facilitates the connection of a guidewire to other devices (e.g., interface cables, catheters) in an environment protected from outside contaminants. Maintaining the cleanliness of the guidewire tip ensures optimal connectivity and signal transmission. The apparatus also reduces the need to extensively handle the guidewire in order to connect it to an interface or place a catheter over it. This is particularly important due to the fragile nature of the guidewire.

The concierge is ideal for optical (OCT) and ultrasound (IVUS) imaging systems due to the possibility of debris interfering with a clean connection. The disclosed concierge device can further be used in any procedure where it is desired to place a quick exchange catheter over a guidewire tip while simultaneously avoiding outside contamination.

In certain aspects, the invention provides a device for an intravascular procedure. The device has a hollow body with a lumen therein, an exit port at a distal end of the device, and a plurality of access ports at a proximal end of the device. A guidewire, a catheter, and a cable are locked into the device. The device is configured so that the catheter can be inserted over the guidewire, the guidewire can be removed from the catheter, and the cable can be inserted into the catheter all within the device. A connection between the guidewire and the cable may be broken within the device. Preferably, the guidewire, the catheter, and the cable are locked into the device such that they are never removed during the useful life of the device, the guidewire, the catheter, and the cable. The device may optionally include a collet (e.g., to grasp and lock the guidewire), a switcher flap (e.g., to close one of the ports when the guidewire is moved away from the port), a ferrule (e.g., to retain the cable therein), or a combination thereof. In some embodiments, the cable provides an intravascular imaging probe.

In related aspects, the invention provides a method for performing an intravascular procedure by inserting a guidewire into tissue within a patient and threading a catheter over a proximal end of the guidewire within a device. The device has a hollow body with a lumen therein, and an exit port at a distal end of the device, and a plurality of access ports at a proximal end of the device. The method includes removing the guidewire from the catheter within the device and inserting a cable into the catheter, also within the device. The method may optionally include retaining the cable within the device via a ferrule, closing one of the access ports with a switcher flap when the guidewire is moved away from the port, preventing the guidewire from leaving the device via the exit port through the use of a collect within the device that locks onto the guidewire, performing intravascular imaging with the cable while it is extended through the device (e.g., OCT, IVUS, or photoacoustic imaging), or a combination thereof.

In some embodiments, the concierge is pre-configured with the guidewire and the interface cable, with the guidewire tip and distal terminus of the interface cable already connected inside the concierge. The operator then disconnects the guidewire from the interface cable (all within the concierge), threads a catheter through a catheter-receiving prong of the concierge and slips the catheter over the guidewire.

### BRIEF DESCRIPTION OF THE DRAWINGS

FIG. 1 shows a guidewire within a concierge device of the invention.

FIG. 2 shows inserting a guidewire through the device.

FIG. 3 shows locking a guidewire into the device.

FIG. 4 shows an interface cable coupled to a guidewire by a connector.

FIG. 5 shows introducing a catheter to the device.

FIG. 6 shows inserting the catheter over the guidewire.

FIG. 7 shows removing a guidewire.

### DETAILED DESCRIPTION

The invention provides a concierge device that receives a guidewire and keeps a tip of the guidewire with in the device at all times. The device also allows a catheter to be slipped over the guidewire, allows the guidewire to be removed from the catheter, and allows a cable of a interventional device to be inserted into the catheter all within the device. Preferably no part of the connecting components are exposed to the outside ambient atmosphere during the described connections, severing of connections, and insertions. The guidewire tip and other parts are never exposed to the outside environment once an intravascular catheterization procedure is underway. The guidewire, once inside the concierge device, can then be connected to an interface cable or sheathed inside a catheter, all inside the concierge device.

FIG. 1 shows a guidewire 101 within a concierge device 105 of the invention. Interface cable 121 can be seen extending from a port of device 105.

FIG. 2 shows inserting a guidewire 101 through device 105. Switcher flap 137 is moved aside, allowing guidewire 101 to extend through device 105 and out a distal port. Guidewire 101 extends through collet 109. Here, collet 109 is shown in an open position. Ferrule 133 attached to a distal portion of interface cable 121 keeps interface cable 121 within device 105. A proximal portion of interface cable 121 passes to instrument 125.

FIG. 3 shows guidewire 101 locked the device 105. Guidewire 101 has traveled to its distal-most position, and collet 109 has locked to guidewire 101, preventing guidewire 101 from exiting device 105. Switcher flap 137 has returned to a closed position. A catheter 145 is at the ready, inserted into a port of device 105.

FIG. 4 shows interface cable 121 coupled to guidewire 101 by a connector 149 (collet 109, ferrule 133, or both can be a style that does “double duty” as connector 149, or an independent connector 149 can be used). Connector 149 may provide a fixed or a slidable coupling. Where connector 149 provides a slidable coupling, a surgeon can push interface cable 121 to the location of guidewire 101 within a patient's body. Interface cable 121 can be any suitable cable known in the art. For example, interface cable 121 may be a medical imaging catheter. Interface cable 121 could be a probe for intravascular imaging by, e.g., IVUS, OCT, or photoacoustic imaging.

In some embodiments, a device 105 is provided with an internal connection pre-made. A surgeon may wish to use, for example, a guidewire 101 that is already connected to interface cable 121.

FIG. 5 shows catheter 145 being inserted into device 105. In some embodiments, once guidewire 101 has been guided to a region of interest within a patient's tissue, catheter 145 is threaded over guidewire 101 and also inserted to the region of interest. As shown in FIG. 5, while catheter 145 is pushed forward (in a distal direction), guidewire 101 can be brought in a proximal direction if desired, so that guidewire 101 enters catheter 145 in a controlled fashion (e.g., held straight and true by device 105).

FIG. 6 shows inserting catheter 145 over guidewire 101. Catheter 145 may include a connector lock 147 that can connect to either guidewire 101, interface cable 121, or both. Connector lock 147 may itself be a collet or it may include a channel or groove for receiving interface cable 121. Collet 109 may independently be used to retain guidewire 101 within device 105.

FIG. 7 shows removing a guidewire 101. As shown in FIG. 7, catheter 145 has been inserted into the tissue of the patient to the location reached by guidewire 101. Catheter 145 is thus in position for access by interface cable 121. Guidewire 101 is withdrawing through its port in device 105. A proximal portion of catheter 145 may be held in a desirable orientation by device 105 such that advancing interface cable 121 causes interface cable 121 to be inserted into catheter 154.

It will be appreciated that device 105 is operable in a number of intravascular procedures, and allows for the switching between guidewire 101, cable 121, catheter 145, and others in an entirely enclosed space. Additionally, connection between guidewire 101, cable 121, catheter 145, and others can be made or separated entirely within the enclosed space of device 105. Device 105 may be used in intravascular imaging (e.g., IVUS, OCT, or photoacoustic imaging) or other catheterization procedure. Device 105 may be used, for example, in an angioplasty procedure.

For angioplasty and similar procedures, catheter 145 is capable of being delivered over a guidewire 101. In some embodiments, catheter 145 is an intravascular balloon catheters as is used for such procedures as balloon angioplasty, or percutaneous transluminal coronary angioplasty (PTCA). Catheter 145 generally has an elongate tubular shaft with proximal portion and distal portion, and may include one or more passages or lumens. Use of pliable materials provides flexibility or maneuverability, allowing a catheter to be guided to a treatment site in a patient's blood vessels. Preferably, a catheter of the invention has enough stiffness to allow it to be pushed to a target treatment site, and accordingly, an ability to optimize a balance of pliability versus stiffness or pushability is beneficial to medical use. In certain embodiments, catheter 145 includes a stiffening wire or coil, or a reinforcement coil, to aid in transmitting the vibration form the proximal end to the distal end. Additionally, a shaft of the catheter can be provided that is capable of transmitting torque along an axis of the shaft. Devices for cardiovascular intervention are discussed in U.S. Pat. Nos. 6,830,559; 6,074,362; and U.S. Pat. No. 5,814,061, the contents of each of which are incorporated by reference.

Catheter 145 could optionally include an angioplasty balloon or other interventional device at distal portion to expand or dilate blockages in blood vessels or to aid in the delivery of stents or other treatment devices. Blockages include the narrowing of the blood vessel called stenosis.

Typically, a catheter 145 will include a guidewire lumen so that the catheter may be advanced along a guidewire. Guidewire lumen in a balloon catheter is described in U.S. Pat. No. 6,022,319 to Willard. Catheter 145 may include any suitable material such as, for example, nylon, low density polyethylene, polyurethane, or polyethylene terephthalate (PET), or a combination thereof (e.g., layers or composites). An inner surface of a guidewire lumen may include features such as a silicone resin or coating or a separate inner tube made, for example, of preformed polytetrafluoroethylene (PTFE). The PTFE tube may be installed within the catheter shaft by sliding it into place and then shrinking the catheter shaft around it. This inner PTFE sleeve provides good friction characteristics to the guidewire lumen, while the balance of the catheter shaft can provide other desired qualities. Other suitable materials for use in catheter 145 or an inner tube portion thereof include high density polyethylene (HDPE) or combinations of material, for example, bonded in multiple layers. Guidewire 109 can be used to deliver a catheter carrying treatments, such as angioplasty balloons or stents. Intravascular procedures are described in U.S. Pat. No. 8,361,097 to Patel; U.S. Pat. No. 8,298,149 to Hastings; and U.S. Pat. No. 5,713,848 to Dubrul, the contents of each of which are incorporated by reference.

Catheter 145 may include coaxial tubes defining separate inflation and guidewire lumens, for example, along a portion of, or an entirety of, a length of catheter 145. A plurality of lumens may be provided in parallel configuration or coaxial at one point and parallel at another, with a twisting/plunging portion to affect a transition between the parallel segment and the coaxial segment (see., e.g., U.S. Pat. No. 7,044,964). Other possible configurations include one or more of a guidewire tube or guidewire lumen disposed outside of the balloon. Or the guidewire tube may be affixed to and extend along the wall of the balloon. In some embodiments, a proximal end of guidewire 101 is mounted in a torquer device. Any torquer device may be used. For example, a handle member may be fixed onto proximal end of guidewire 101 by welding, adhesives, clamps, or other suitable means. In some embodiments, the torque device comprises a pin vise.

In some embodiments, guidewire 101 is used for crossing a chronic total occlusion (CTO). As a distal portion of guidewire 101 approaches a CTO, a physician can optionally view the site on a monitor of an associated medical imaging instrument (e.g., via an imaging catheter 121). Such vascular intervention procedures by catheter are often performed in specialized clinical environments known as cath labs. The catheterized intervention procedures described herein may be performed with associated imaging procedures (e.g., using IVUS and OCT instruments). Exemplary IVUS methods are discussed in U.S. Pat. No. 8,289,284; U.S. Pat. No. 7,773,792; U.S. Pub. 2012/0271170; U.S. Pub. 2012/0265077; U.S. Pub. 2012/0226153; and U.S. Pub. 2012/0220865. OCT systems and methods are described in U.S. Pub. 2011/0152771; U.S. Pub. 2010/0220334; U.S. Pub. 2009/0043191; U.S. Pub. 2008/0291463; and U.S. Pub. 2008/0180683, the contents of each of which are hereby incorporated by reference in their entirety.

In some embodiments, catheter 145 is used to deliver an angioplasty balloon. Generally, a balloon will include a flexible, inelastic material designed to expand. By this type of expansion, a balloon may impose pressures of several atmospheres to expand the stenosis or may be used to deploy a stent. After the balloon has been expanded, it is then deflated and removed from the patient, allowing improved blood flow through the vessel. Suitable materials may include polyvinyl chloride (PVC), nylon, polyethylene terephthalate (PET), polybutylene terephthalate (PBT) and copolyesters, polyether-polyester block copolymers, polyamides, polyurethane, poly(ether-block-amide) and the like. Balloons are described in U.S. Pat. No. 7,004,963; U.S. Pub. 2012/0071823; U.S. Pat. No. 5,820,594; and U.S. Pub. 2008/0124495, the contents of each of which are incorporated by reference. Balloon catheters are described in U.S. Pat. No. 5,779,731 and U.S. Pat. No. 5,411,016, incorporated by reference.

In some embodiments, the balloon includes artificial muscle (electro-active polymer). Electro-active polymers exhibit an ability to change dimension in response to electric stimulation. The change may be driven by electric field E or by ions. Exemplary polymers that respond to electric fields include ferroelectric polymers (commonly known polyvinylidene fluoride and nylon 11, for example), dielectric EAPs, electro-restrictive polymers such as the electro-restrictive graft elastomers and electro-viscoelastic elastomers, and liquid crystal elastomer composite materials. Ion responsive polymers include ionic polymer gels, ionomeric polymer-metal composites, conductive polymers and carbon nanotube composites. Common polymer materials such as polyethylene, polystyrene, polypropylene, etc., can be made conductive by including conductive fillers to the polymer to create current-carrying paths. Many such polymers are thermoplastic, but thermosetting materials such as epoxies, may also be employed. Suitable conductive fillers include metals and carbon, e.g., in the form of sputter coatings. Electro-active polymers are discussed in U.S. Pat. No. 7,951,186; U.S. Pat. No. 7,777,399; and U.S. Pub. 2007/0247033, the contents of each of which are incorporated by reference.

In some embodiments, guidewire 101 and/or catheter 145 is used to deliver a stent. Any suitable stent may be used with device 101. One exemplary device for stent is the Palmaz-Schatz stent, described, for example, in U.S. Pat. No. 4,733,665. Suitable stents are described in U.S. Pat. No. 7,491,226; U.S. Pat. No. 5,405,377; U.S. Pat. No. 5,397,355; and U.S. Pub. 2012/0136427, the contents of each of which are expressly incorporated herein by reference. Generally, a stent has a tubular body including a number of intersecting elongate struts. The struts may intersect one another along the tubular body. In a non-deployed state, the tubular body has a first diameter that allows for delivery of the stent into a lumen of a body passageway. When deployed, the stent has a second diameter and deployment of the stent causes it to exert a radially expansive force on the lumen wall. Methods of using stents are discussed in U.S. Pat. No. 6,074,362; U.S. Pat. No. 5,158,548; and U.S. Pat. No. 5,257,974, the contents of each of which are incorporated by reference. In some embodiments, stent 161 includes a shape-retaining or shape memory material such as nitinol and is self-expanding and thermally activatable within a vessel upon release. Such devices may automatically expand to a second, expanded diameter upon being released from a restraint. See, e.g., U.S. Pat. No. 5,224,953, the contents of which are incorporated herein by reference.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A device for an intravascular procedure, the device comprising:
a hollow body with a lumen therein, and an exit port at a distal end of the device; and
a plurality of access ports at a proximal end of the device, wherein the device is configured so that a catheter can be inserted over a guidewire, the guidewire can be removed from the catheter, and a cable can be inserted into the catheter all within the device.

2. The device of claim 1, wherein the guidewire, the catheter, and the cable are locked into the device such that they are never removed during the useful life of the device, the guidewire, the catheter, and the cable.

3. The device of claim 2, wherein the plurality of access ports comprises at least 3 access ports.

4. The device of claim 3, further comprising a switcher flap that closes one of the plurality of access ports when the guidewire is moved away from the port.

5. The device of claim 4, further comprising a collet disposed therein that grasps and locks the guide wire to prevent it from exiting the device.

6. The device of claim 2, wherein the cable comprises an intravascular imaging probe.

7. The device of claim 2, further comprising a cap to cover a proximal tip of the guidewire.

8. The device of claim 2, wherein the guidewire is connected to the cable by a ferrule.

9. The device of claim 8, wherein connection between the guidewire and the cable can be broken within the device.

10. The device of claim 2, further comprising a ferrule to retain the cable in the device.

11. A method for performing an intravascular procedure, the method comprising:
inserting a guidewire into tissue within a patient;
threading a catheter over a proximal end of the guidewire within a device comprising a hollow body with a lumen therein, and an exit port at a distal end of the device, and a plurality of access ports at a proximal end of the device;
removing the guidewire from the catheter within the device; and
inserting a cable into the catheter within the device.

12. The method of claim 11, further comprising retaining the cable within the device via a ferrule.

13. The method of claim 11, further comprising closing one of the access ports with a switcher flap when the guidewire is moved away from the port.

14. The method of claim 11, further comprising preventing the guidewire from leaving the device via the exit port through the use of a collect within the device that locks onto the guidewire.

15. The method of claim 11, further comprising performing intravascular imaging with the cable while it is extended through the device.

16. The method of claim 15, wherein the intravascular imaging comprises OCT, IVUS, or photoacoustic imaging.
